(19) United States
(12) Patent Application Publication
(10) Pub. No.: US 2022/0136046 A1
(43) Pub. Date: May 5, 2022

(54) DETECTION AND ANTIBIOTIC RESISTANCE PROFILING OF MICROORGANISMS

(71) Applicant: ST GEORGE'S HOSPITAL MEDICAL SCHOOL, London (GB)

(72) Inventors: Tariq SADIQ, London, Greater London (GB); Liqing ZHOU, London, Greater London (GB)

(21) Appl. No.: 17/436,157

(22) PCT Filed: Mar. 3, 2020
(86) PCT No.: PCT/GB2020/050506

§ 371 Date: Sep. 3, 2021

(30) Foreign Application Priority Data
Mar. 4, 2019 (GB) 1902887.7

(51) Int. Cl. C12Q 1/6869; C12Q 1/689; C12Q 1/686

(52) U.S. Cl. CPC C12Q 1/6869; C12Q 1/686; C12Q 1/689

(57) ABSTRACT

The invention is in the field of the identification of microorganisms and profiling of the antimicrobial resistance. In particular, a method has been identified which allows the simultaneous identification and antimicrobial resistance profiling. Accordingly, the method allows for rapid diagnosis of an microbial infection.

### FIELD OF THE INVENTION

The present invention relates to the detection of microorganisms and/or profiling of the antimicrobial resistance of the microorganism. Preferably, the present invention relates to the simultaneous detection and antimicrobial profiling of a microorganism using a single target gene. The invention also provides kits for use in such methods.

### BACKGROUND OF THE INVENTION

Sexually transmitted infections (STIs), caused by Neisseria gonorrhoeae (NG), Chlamydia trachomatis (CT), Trichomonas vaginalis (TV) and Mycoplasma genitalium (MG), are a significant public health concern. A key factor for reducing the burden and transmission of STIs is shortening duration of infectiousness, which can be in part achieved by treating STIs immediately at diagnosis.

Antimicrobial resistance (AMR) is an increasing global health challenge. A key element in preventing AMR spread, enabling appropriate treatment for infected patients and antibiotic stewardship is the development of accurate diagnostics that accurately predict antibiotic susceptibility. This is particularly important as the use of culture has reduced significantly, and STIs are conventionally treated at the point of diagnosis or presumptively. Molecular diagnostics for pathogen detection have transformed clinical care for patients over the past two decades and novel target-based nucleic acid amplification technologies (NAATs) can not only identify those patients with infections but those with drug resistant strains, where relevant. Near patient or point of care (PoC) versions for some infections have been developed whilst for others are in development.

In the UK and US, gonorrhoea treatment guidelines recommend empirical therapy only with antibiotics that are predicted, by local phenotypic surveillance, to be effective against >95% of circulating strains. Historically, AMR within NG has become prevalent against multiple classes of antibiotics following their introduction to national recommendations, in-turn precluding their use for empirical therapy. Currently, combination therapy with ceftriaxone, an expensive, injectable single dose “extended spectrum cephalosporin”, and oral single dose azithromycin is recommended.

However, most circulating strains of NG still remain susceptible to most antibiotics in the UK, including ciprofloxacin, a cheap, simple to use, single dose and effective fluoroquinolone. Until those susceptible strains are identifiable as such at the point of treatment, ceftriaxone and azithromycin must be used. However, emergence of ceftriaxone resistance has now been increasingly reported threatening to make immediate empirical therapy for gonorrhoea practically undeliverable.

CT, TV, MG and NG can have similar clinical presentations. Recent studies have observed high rates of AMR to macrolides within MG which, like CT, is a frequent cause of nongonococcal urethritis (NGU) in men and cervicitis in women but is not routinely tested for, and often remains undiagnosed. Empirical therapy of NGU/cervicitis is usually aimed at preferential treatment of CT with either doxycycline or azithromycin, both increasingly ineffective against MG. A significant proportion of MG strains are still susceptible to azithromycin, and genotypic markers of macrolide resistance known. For TV, in the absence of positive microscopy, itself a sub-optimal diagnostic test for TV, empirical therapy with metronidazole is not usually given in UK, unless epidemiologically, but in other parts of the world with high TV prevalence, treatment for TV is often given syndromically.

Generally, widespread use of antibiotics can promote AMR through selection pressure and it may be possible to slow development of multi-drug resistance by limiting use of the range of antibiotics to which the causative pathogen is susceptible. Thus, there is benefit in diagnosing infection rapidly as well as knowing if the diagnosed pathogen is susceptible or resistant to antibiotic treatment

In clinical microbiology diagnostic laboratories, the gold standard method for the detection and antibiotic resistance profiling of pathogenic microorganisms in patients suspected of infections is culture and susceptibility testing. However, sensitivity of this technique is affected by treatment of the patient with antibiotics, low abundance of the microorganism (especially bacteria) and non-cultivable organisms. Moreover, it takes usually 2 to 5 days to obtain a result from culture, which is often late to initiate proper antibiotic therapy, particularly for the treatment of sexually transmitted infections, where populations are vulnerable and hard to reach.

As an alternative to culture, NAAT-based detection of microorganisms can provide rapid results, and high specificity and sensitivity. Although NAAT-based testing is currently used for rapid diagnosis of STIs, its use in determination of antibiotic susceptibility of infectious agent is limited. There are some developments which detect specific mutations in genes responsible for antibiotic resistance, such as mutations in gyrA gene. However, single nucleotide polymorphism (SNP) or gene-based AMR tests are challenged by the continuous evolution of pathogens selecting for new mutations in existing genes as well as for novel mechanisms of resistance. Additionally, often, due to there being multiple and different genetic mutations/changes that are responsible for antibiotic resistance, it is not technically practical to test for each of them to determine AMR bacterial strains using NAATs.

WO 2016/203267 describes a method to detect the wild type gene instead of mutant to narrow the use of antibiotic treatment. US 2014/030712 used a genomic approach for the identification of biomarkers for antibiotic resistance and susceptibility in clinical isolates of bacterial pathogens. These approaches are not appropriate for use in clinical settings for point of care testing. There is an urgent need for a diagnostic platform to diagnose infections as well as profile antibiotic resistance simultaneously and rapidly.

Whole genome sequencing (WGS) using next generation sequencing platforms may address this challenge to some degree, and give added value in identifying phylogenetic relationships in identified infections. However WGS, can be constrained by the need to culture or to use nucleic acid capture techniques, which are themselves limited by turn-around-time and cost, to be suitable for near patient applications.

Therefore, it was an objective of the present invention to provide a method to overcome the limitations associated with current techniques. In particular, the present inventors used a targeted PCR, followed by long read and single molecule sequencing of a single gene target, to simultaneously characterise the identity and antibiotic resistance profile of the microorganism present in clinical samples.

### SUMMARY OF THE INVENTION

Accordingly, the present invention provides a method for simultaneously detecting a microorganism and for profiling antimicrobial resistance of the microorganism, comprising:

(a) preparing DNA from a sample suspected of containing the microorganism;

(b) conducting targeted amplification of a gene from the microorganism, wherein the gene is responsible for antimicrobial resistance and can identify the microorganism;

(c) sequencing the amplified products obtained in step (b) using long-read sequencing;

(d) comparing the sequence to reference sequences known to be susceptible or resistant to the antimicrobial; and

(e) identifying the microorganism and profiling the antimicrobial resistance of the microorganism.

The present invention also provides a method for identifying a microorganism, comprising:

(a) preparing DNA from a sample suspected of containing the microorganism;

(b) conducting targeted amplification of a gene from the microorganism, wherein the gene can identify the microorganism;

(c) sequencing the amplified products obtained in step (b) using long-read sequencing;

(d) comparing the sequence to reference sequences; and

(e) identifying the microorganism.

Furthermore, the invention provides a method for profiling the antimicrobial resistance of a microorganism, comprising:

(a) preparing DNA from a sample containing the microorganism;

(b) conducting targeted amplification of a gene from the microorganism, wherein the gene is responsible for antimicrobial resistance;

(c) sequencing the amplified products obtained in step (b) using long-read sequencing;

(d) comparing the sequence to reference sequences; and

(e) profiling the antimicrobial resistance of the microorganism.

The invention also provides a kit for use in the method of any one of the preceding methods, comprising reagents for amplification of one or more target genes and instructions for use.

### BRIEF DESCRIPTION OF THE FIGURES

FIG. 1. Diagram showing a method of the invention

FIG. 2. DNA preparation from a clinical sample. Lane M: DNA size marker; lane 1: DNA preparation from a clinical sample FIG. 3. Targeted PCR amplicons from bacterial genomic DNA on 1% agarose gel. Lane M: DNA size marker; lane 1: negative control; lane 2: N. gonorrhoeae; lane 3: C. trachomatis; lane 4: M. genitalium; lane 5: N. gonorrhoeae and C. trachomatis; lane 6: N. gonorrhoeae and M. genitalium; lane 7: C. trachomatis and M. genitalium; lane 8; N. gonorrhoeae, C. trachomatis and M. genitalium.

FIG. 4. Targeted PCR amplicons from different clinical samples on 1% agarose gel.

1) Samples

Lane M: DNA marker; lanes 1 and 3: sample 2133; lanes 2 and 4: sample 2014; lane 5: sample 2072; lanes 6 and 10: sample 1188; lane 7: sample 2065; lane 8: sample 1006; lane 9: sample 1070.
2) PCR gene target

Lanes 1 and 2: gyrA of N. gonorrhoeae; lane 3 and 4: omp1 of C. trachomatis; lanes 5, 6 and 7: 23S rRNA of M. genitalium; lanes 8, 9 and 10: ntr6 of T. vaginalis.

FIG. 5. Nanopore sequencing WIMP report showing the read counts aligning to Neisseria gonorrhoeae (see text).

FIG. 6. Nanopore sequencing ARMA report showing the read counts aligning to Neisseria gonorrhoeae genes conferring resistance to fluoroquinolone.

FIG. 7. Distribution of sequencing read counts of clinical swab samples

FIG. 8. Pathogen specific reads/classified read count ratios of clinical swab samples in singlex PCR-targeted long read sequencing.

FIG. 9. Pathogen specific reads/classified read count ratios and Ct values of clinical swab samples (samples in which infection was not detected are outlined)

FIG. 10. Pathogen specific reads/read counts classified ratios of clinical swab samples sequenced in triplex PCR targeted long read sequencing

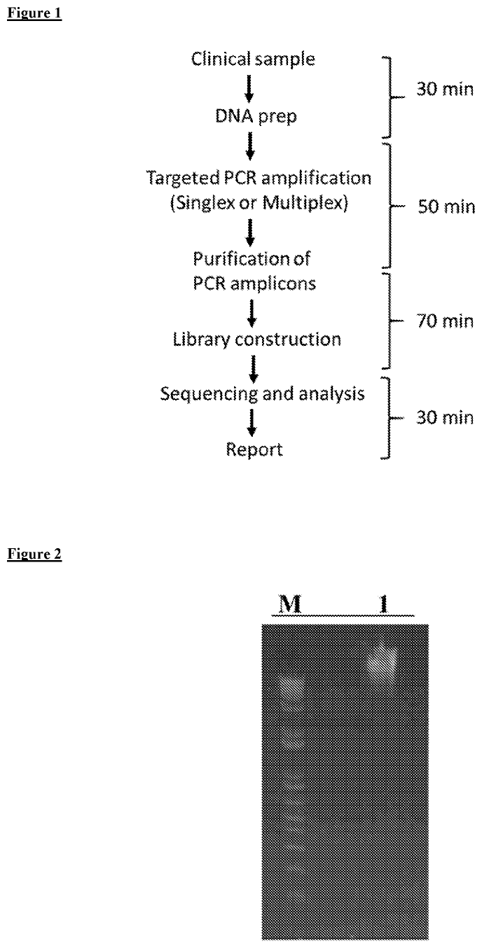

### BRIEF DESCRIPTION OF THE SEQUENCE LISTING

SEQ ID NOs: 1-33 show sequences used in the examples.

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “an amino acid sequence” includes two or more such sequences, and the like.

All publications, patents and patent applications cited herein, whether supra or infra, are hereby incorporated by reference in their entirety.

The present invention provides a method for simultaneously detecting a microorganism and for profiling antimicrobial resistance of the microorganism, comprising:

(a) preparing DNA from a sample suspected of containing the microorganism;

(b) conducting targeted amplification of a gene from the microorganism, wherein the gene is responsible for antimicrobial resistance and can identify the microorganism;

(c) sequencing the amplified products obtained in step (b) using long-read sequencing;

(d) comparing the sequence to reference sequences known to be susceptible or resistant to the antimicrobial; and

(e) identifying the microorganism and profiling the antimicrobial resistance of the microorganism.

The present invention thus provides a method for identifying the presence of a particular microorganism in a sample. The method may in particular be used to diagnose a microbial infection.

The present invention allows the antimicrobial resistance of the microorganism to be profiled, i.e. determination of whether the microorganism is susceptible or resistant to a particular antimicrobial.

The invention provides for simultaneous identification of the microorganism and profiling of the antimicrobial resistance. In other words, the method of the invention provides results which both identify the microorganism and the antimicrobial resistance. In particular, the invention allows for both identification of a microorganism and for profiling of the antimicrobial resistance of the microorganism based on analysis of a single target gene.

The microorganism identified using the method is not limited, but is typically a bacterium, parasite or fungus. The methods of the invention are preferably used in relation to microorganisms responsible for sexually transmitted infections (STIs). Microorganisms responsible for STIs which may be detected using the methods of the invention include Neisseria gonorrhoeae (NG), Chlamydia trachomatis (CT), Trichomonas vaginalis (TV) and Mycoplasma genitalium (MG).

An antimicrobial may be for example an antibiotic, an anti-parasitic or an anti-fungal agent. Such antimicrobial agents for treating an infection with a particular microorganism would be well known to the skilled person. For example, NG may be treated with cephalosporins and fluoroquinolones, such as ceftriaxone—given as an injection—in combination with either azithromycin or doxycycline. Azithromycin or doxycycline may be used for the treatment of CT, metronidazole is commonly used to treat TV and azithromycin is commonly used to treat MG.

Before conducting the steps outlined above, a sample is typically obtained from a patient suspected of having an infection. The sample may be any appropriate sample type for the suspected infection. Such sample types would readily be known to skilled healthcare personnel. Typically, samples may be a blood, serum, plasma, urine, saliva or faeces sample. For STIs, urine samples are commonly used. Samples may also be obtained from swabbing. For example, a vaginal or anal swab may be appropriate when testing for an STI. In some instances, multiple sample types may be screened in the methods of the invention.

The subject may be, for example, a domestic or livestock animal, but is typically a human. The subject may be suspected of having such an infection because the subject presents with symptoms of the infection. These symptoms would be well known, particularly to a medical or veterinary practitioner. For example, a female subject suspected of having CT may present with pain when urinating, vaginal discharge, pain in the abdomen pelvis, pain during sex, bleeding after sex or bleeding between periods. A male subject may present with pain when urinating, discharge, burning in the urethra or pain in the testicles.

In some instances, the sample may be obtained from an alternative source, such as by swabbing a surface suspected of harbouring the microorganism.

It is not necessary to treat the sample in any particular way prior to testing using the methods of the invention but if desired steps may be taken to preserve the sample in the time taken from removal of the sample from the subject and testing using the methods of the invention. For example, fresh blood samples may be collected in sterile tubes containing heparin prior to testing, whilst swabs may be collected into transport medium.

Once the sample has been obtained, DNA from the sample is prepared. DNA is typically isolated from the sample and purified. Methods and kits for isolating DNA are well known in the art and are described in the Examples below. The skilled person would readily be able to select a suitable technique for preparing DNA based for example on the nature of the sample (e.g. the size of the sample and whether the sample is fresh or frozen).

Once the DNA has been prepared, targeted amplification of a gene from the microorganism is conducted. The amplification of the target gene is selective in that the target DNA is amplified under conditions where non-target DNA is not amplified.

Genes responsible for antimicrobial resistance and which can identify a microorganism would be well known to the skilled person. For example, numerous studies have been conducted investigating antimicrobial resistance in NG, CT, TV and MG.

Mutations in the gyrA gene are known to be responsible for fluoroquinolone resistance in NG (Knapp et al., Emerg, Infect. Dis. 1997; 3:33-39), mutations in 23S rRNA are known to be responsible for macrolide resistance in MG (Jensen et al., Clin Infect Dis. 2008; 47(12):1546-53) and mutations in the nitroreductase genes ntr4 and ntr6 are known to be responsible for metronidazole resistance in TV whilst the ntr6 mutation may have clinical utility in identifying metronidazole-resistant TV due to its higher prevalence in resistant isolates than in susceptible isolates (Paulish-Miller et al., Antimicrob Agents Chemother. 2014 May; 58(5): 2938-2943). Methods of the invention may therefore involve targeted amplification of one or more of these genes. However, the methods are not limited to these target genes. The skilled person would readily be able to identify other appropriate target genes based on the information available for a particular microorganism to realize the purpose of the invention, for example, the parC gene of NG can also be chosen for use to simultaneously identifying NG and profiling its antibiotic resistance.

The gene is also capable of identifying the microorganism. In other words, the sequence of the gene is specific to a particular microorganism. Sequencing of the gene thus allows for the microorganism to be identified, provided that a sufficient length of the gene, preferably equal to or larger than 500 bp, is sequenced to distinguish over related sequences from other microorganisms. In some instances, a length equal to or larger than 750 bp or 1000 bp could be sequenced.

Targeted amplification of a gene may be conducted by any known method, such as Polymerase Chain Reaction (PCR), Loop Mediated Isothermal Amplification (LAMP), Nucleic Acid Sequence Based Amplification (3 SR or NASBA), strand displacement amplification (SDA), Rolling Circle Amplification (RCA), ligase chain reaction (LCR) and Recombinase Polymerase Amplification (RPA), but is preferably conducted by PCR. The skilled person would readily be able to design primers appropriate for selective amplification of a particular gene (the primers are specific for the target gene DNA). Primers for amplification of gyrA in NG are described below in the Examples, as are primers for amplification of the omp1 gene of CT, the 23S rRNA gene of MG and the ntr6 gene of TV.

The primers are typically appropriate to allow for amplification and sequencing of the entire target gene. In some instances, the primers may allow for amplification and sequencing of a smaller region, provided enough of the gene flanking the antimicrobial resistance determining region is amplified in order for the microorganism to be identified, typically, a region of at least 500 bp. The skilled person would readily be able to determine appropriate primers based on the known sequence of the microorganism. Such known sequences are discussed below with regards to “reference sequences”.

The PCR may either be singlex or multiplex PCR (for example triplex PCR). Multiplex PCR refers to the use of PCR to amplify several different DNA sequences of one or more microorganisms simultaneously using multiple primer sets. Once again, the skilled person would readily be able to design appropriate primers.

Multiplex formats can be easily applied in clinical practice and singlex formats may be advantageous where more targets are to be amplified and sequenced. As shown in the examples, triplex PCR may for example target NG, CT and MG.

Greater rapidity may be gained by using fast isothermal amplification methods.

The PCR amplicons from different samples may be barcoded for analysing more than one sample in one sequencing run. The amplification may therefore utilise barcoded primers.

After PCR, the PCR amplicons are typically pooled and purified. Methods and kits for purifying PCR products are well known in the art, such as those described in the Examples below.

The next step in the method is that the amplified products are sequenced using long-read sequencing technologies (also known as “third generation”), which use different methods to read much longer stretches of DNA sequence than earlier sequence methods. Examples of long read sequencing technologies include PacBio and nanopore sequencing. The methods of the invention typically utilise nanopore sequencing (such as the sequencing protocols developed by Oxford Nanopore Technologies), because of its potential for rapidity but can also be adapted to the PacBio platform. Any long read or long fragment sequencing techniques could also potentially be used in the methods of the invention. However, long read sequencing techniques as described above are preferably used.

Prior to sequencing, the amplified products may be processed in any appropriate manner for the selected sequencing technique.

The sequences are then compared bioinformatically to reference sequences. The reference sequences are from microorganisms known to be either susceptible or resistant to a particular antimicrobial. Comparison to the reference sequences therefore allows the microorganism to be identified and the antimicrobial resistance to be determined.

The reference sequences are typically obtained from a database, such as NCBI Reference Sequence Database (RefSeq) and The Comprehensive Antibiotic Resistance Database (CARD). For example, the Oxford Nanopore Technology “What's in my pot (WIMP)?” application uses RefSeq to allow for real time identification of a particular sequence, and antimicrobial resistance mapping application (ARMA) uses the CARD for real time profiling of antimicrobial resistance.

As discussed in the Examples section below, the inventors have determined that nanopore long read sequencing can rapidly identify the presence and profile the antibiotic resistance of NG, MG and TV in pathogen-positive clinical samples and in real time using gyrA, 23S rRNA and ntr6, respectively. The inventors also detected the presence of CT in positive samples using the omp1 gene. The methods of the invention can therefore potentially be used in clinical setting as a point of care test. The inventors have in particular demonstrated that such sequencing achieved thousand times coverage of each target gene within a short time frame using the DNA materials obtained from the targeted PCR, compared with that without targeted PCR amplification (see table 1). Accordingly, the method overcomes shortcomings caused by direct sequencing on the DNA preparations from clinical samples and provides a rapid diagnosis of infections and simultaneous profile of the antimicrobial resistance.

In summary, the invention provides a single gene approach for both identifying a microorganism and for determining the antimicrobial resistance of the microorganism. Such a single gene approach is viable with long read sequencing because the long read sequencing provides much greater specificity than other sequencing approaches. For example, resistance of NG to fluoroquinolones is conventionally determined using sequencing of a short region (e.g. 20 nucleotides). Whilst this short read is sufficient to determine whether the NG is sensitive or resistance to the fluoroquinlone, there would not be enough specificity to identify that the microorganism is NG (compared with other Neisseria species). Long read sequencing allows a large fragment or entire length of the gyrA gene to be read and hence both the identity of the microorganism to be determined and the antimicrobial resistance profiled. This avoids the need to use two or more target sequences and therefore provides a more rapid diagnosis.

In addition to the above, the invention also provides a method for identifying a microorganism. The method comprises:
(a) preparing DNA from a sample suspected of containing the microorganism;
(b) conducting targeted amplification of a gene from the microorganism, wherein the gene is specific to the microorganism;
(c) sequencing the amplified products obtained in step (b) using long-read sequencing;
(d) comparing the sequence to reference sequences; and
(e) identifying the microorganism.

The microorganism and sample may be any of the microorganisms and sample types described above. The DNA may also be prepared from the sample using the methods described above.

In contrast to the method described above, in this aspect a gene specific to the microorganism is amplified (the gene does not also need to be responsible for antimicrobial resistance). The gene may be any gene which, when sequenced, would identify the particular microorganism. The skilled person would readily be aware of such genes.

The amplification, sequencing and comparison to reference sequences may then be carried out as described above.

This method may for example be used to identify a CT infection, particularly using the omp1 gene as the target.

Similarly, the invention provides a method for profiling the antimicrobial resistance of a microorganism, comprising:
(a) preparing DNA from a sample containing the microorganism;
(b) conducting targeted amplification of a gene from the microorganism, wherein the gene is responsible for antimicrobial resistance;
(c) sequencing the amplified products obtained in step (b) using long-read sequencing;
(d) comparing the sequence to reference sequences; and
(e) profiling the antimicrobial resistance of the microorganism.

In this method, the identity of the microorganism is typically already known and the method is used to profile the antimicrobial resistance of the microorganism. Steps (a)-(e) may be carried out as described above.

Once the microorganism has been identified and/or the antimicrobial resistance of the microorganism determined, the microbial infection may then be treated using any appropriate means. For example, if the microorganism is identified as being susceptible to a particular antimicrobial then that antimicrobial may be used in the treatment. If, however, the microorganism is identified as being resistant to an antimicrobial, an alternative antimicrobial can be selected for the treatment.

Finally, the invention provides kits for use in the methods of the invention. The kits typically comprise reagents for amplification of the target gene e.g. primers. By inclusion of such primers the kits may be customised for amplification of DNA from a particular microorganism. The kits may also contain standard reagents for PCR amplification, for example thermostable polymerase, dNTPs, reaction buffer etc. The kits may also contain reagents suitable for preparing the DNA.

In some instances, the kits may also contain means for conducting the long-read sequencing and/or means for obtaining a sample from the patient. The sample type may be any of those discussed above.

Finally, the kits may contain instructions for use.

The following examples are presented below so as to provide those of ordinary skill in the art with a complete disclosure and description of how to make and use the methods and compositions of the invention. The examples are not intended to limit the scope of what the inventors regard as their invention.

### EXAMPLES

### Example 1: DNA Preparation from Clinical Gonorrhoea Urine Samples

DNA was isolated from clinical samples using QIAamp DNA mini kit (Qiagen) according to the manufacturer's instruction, except that the DNA was eluted with 50 μl AE preheated at 65° C. and the filter unit was incubated at 65° C. for 5 min before centrifugation. The eluted DNA was further precipitated with ethanol and dissolved in nuclease free water when used for sequencing library construction. DNA quantification was done using Qubit 3.0 fluorometer (Invitrogen). The size distribution of DNA preparation was checked through electrophoresis on 0.8% agarose E-gel EX (Invitrogen), as shown in FIG. 2.

### Example 2: Design and Synthesis of PCR Primers of Gene Targets

Neisseria gonorrhoeae Gene Target:

Neisseria gonorrhoeae gene gyrA was used for simultaneously identifying N. gonorrhoeae and profiling its antibiotic susceptibility to ciprofloxacin (a fluoroquinolone).

Primers of gyrA without barcode are:

(SEQ ID NO: 1)
NG_gyrA_F: 5′ ATCCGCCACGACCACAAATT
(SEQ ID NO: 2)
NG_gyrA_R: 5′ ATATTGGACAGTGCGACGGC

Primers of gyrA with barcode are:

NG_gyrA_BC_F:
(SEQ ID NO: 3)
5′ TTTCTGTTGGTGCTGATATTGCATCCGCCACGACCACAAATT
NG_gyrA_BC_R:
(SEQ ID NO: 4)
5′ ACTTGCCTGTCGCTCTATCTTCATATTGGACAGTGCGACGGC

Chlamydia trachomatis Gene Target:

Chlamydia trachomatis major outer membrane protein 1 (Omp 1) gene was used for identifying the presence of Chlamydia trachomatis.

Primers of OMP1 without barcode are:

(SEQ ID NO: 5)
CT_OMP1_F: 5′-TTT GCC GCT TTG AGT TCT GCT
(SEQ ID NO: 6)
CT_OMP1_R: 5′-CAATACCG CAA GAT TTT CTA GAT TTC

Primers of OMP1 with barcode are:

CT_OMP1_BC_F:
(SEQ ID NO: 7)
5′ TTTCTGTTGGTGCTGATATTGCTTTGCCGCTTTGAGTTCTGCT
CT_OMP1_BC_R:
(SEQ ID NO: 8)
5′ ACTTGCCTGTCGCTCTATCTTCCAATACCGCAAGATTTTCTAGAT
TTC

Mycoplasma genitalium Gene Target:

Mycoplasma genitalium 23 S ribosomal RNA gene was used for simultaneously identifying Mycoplasma genitalium and profiling its antibiotic susceptibility to macrolide

Primers of 23 S rRNA without barcode are;

Mg 23S_1992F:
(SEQ ID NO: 9)
5′ CCATCTCTTGACTGTCTCGG
Mg 23S_2679R:
(SEQ ID NO: 10)
5′ TCTCGTACTAGAAGCAAAG
Primers of 23S rRNA with barcode are;
Mg 235_BC_1992F:
(SEQ ID NO: 11)
5′ TTTCTGTTGGTGCTGATATTGCCCATCTCTTGACTGTCTCGG
Mg 23S_BC_2679R:
(SEQ ID NO: 12)
5′ ACTTGCCTGTCGCTCTATCTTCTCCTCTCGTACTAGAAGCAAAG

Trichomonas vaginalis Gene Target:

Trichomonas vaginalis nitroreductase family protein (ntr6) was used for simultaneously identifying Trichomonas vaginalis and profiling its antibiotic susceptibility to metronidazole.

Primers of ntr6 without barcode are:

TV_NTR6_F:
(SEQ ID NO: 13)
5′ CTTCATTGAATTTATTCGTTCAAAATT
TV_NTR6_R:
(SEQ ID NO: 14)
5′ TTATTCAATGTATGTAACCTTTCTAA

Primers of ntr6 with barcode are:

TV_NTR6_BC_F:
(SEQ ID NO: 15)
5′TTTCTGTTGGTGCTGATATTGCCTTCATTGAATTTATTCGTTCAA
AATT
TV_NTR6_BC_R:
(SEQ ID NO: 16)
5′ACTTGCCTGTCGCTCTATCTTCTTATTCAATGTATGTAACCTTTCTAA

All the primers were synthesized by Sigma Genosys (Sigma-Aldrich, Dorset, England).

### Example 3: Targeted PCR Amplification

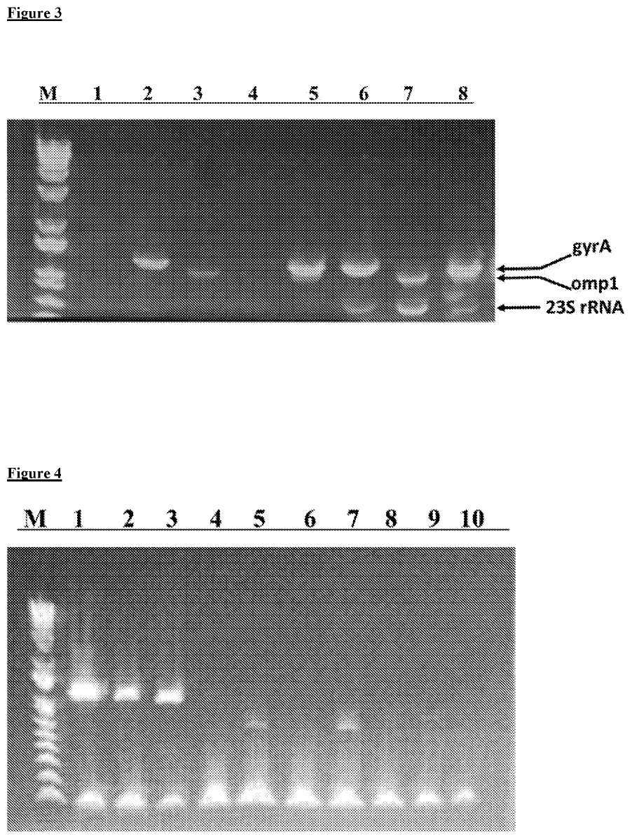

Targeted PCR amplification was performed by using LongAmp Taq 2× Master Mix (New England Biolabs). Each reaction was carried out in 20 μl volume containing 10 LongAmp Taq 2× Master Mix, 50 of forward and revers primer mix with each at 2 μl DNA (20 ng) and 3 μl Nuclease-free water. The following amplification steps were used: 1 cycle of 95° C. for 3 min; 20 cycles of 95° C. 30 sec, 58° C. 30 sec, and 65° C. 1.5 min, 93° C. for 30 sec, 58° C. for 30 sec, and 72° C. for 40 sec; and 1 cycle of 72° C. for 5 min. PCR reactions were checked by running an aliquot on 1% E-gel EX (Invitrogen), and examples are shown in FIG. 3 and FIG. 4.

### Example 4: Purification and Quantification of PCR Amplicons

PCR reactions were combined and the PCR amplicons were purified using Qiagen MinElute Reaction Cleanup Kit according to the manufacturer's instruction, or using Agencourt AMPure XP (Beckman Coulter) according to the following procedure:

1) Bring the AMPure XP beads to room temperature.

2) Transfer the PCR reaction into a 1.5 ml Eppendorf LoBind tube (48-50 μl).

3) Vortex the AMPure XP beads for 30 seconds to make sure that the beads are evenly dispersed.

4) Add 25 μl of AMPure XP beads to each sample and mix by pipetting.

5) Incubate on a rotator mixer for 5 minutes at room temperature.

6) Prepare fresh 80% ethanol in nuclease-free water (500 μl for each sample).

7) Spin down the sample and pellet on a magnet. Keep the tube on the magnet, and pipette off the supernatant.

8) Keep the tube on magnet, and add 200 μl of freshly prepared 80% ethanol without disturbing the pellet.

9) Incubate the tube on the magnetic stand for 30 seconds, and then remove and discard the 80% ethanol carefully.

10) Repeat steps 8-9.

11) Spin down and place the tube back on the magnet. Pipette off any residual 80% ethanol.

12) Keep the tube the magnetic stand with the cap open, and allow the beads to air dry for 1 minute.

13) Remove the tube from the magnetic rack and resuspend pellet in 30 μl nuclease-free water. Incubate for 2 minutes at room temperature.

14) Pellet the beads on magnet until the eluate is clear and colourless.

15) Remove and retain 25 μl of eluate into a clean 1.5 ml Eppendorf DNA LoBind tube.

The PCR amplicons were quantified using Qubit dsDNA BR Assay Kit (Invitrogen).

### Example 5: Rapid Nanopore Sequencing and Real Time Identification of N. gonorrhoeae

Nanopore DNA sequencing was performed on MinION Mk I using SpotON Flow Cell Mk I (R9.4) (Oxford Nanopore Technologies). A rapid sequencing library was prepared using Rapid Sequencing Kit SQK-RAD002 according to the manufacturer's instruction, except that 400 ng input DNA was used. Nanopore sequencing reads were live base-called on a local computer using the MinKNOW protocol script—NC_48Hr_Sequencing_Run_FLO-MIN106_SQK-RAD002.py.

The live basecalled read sequence was uploaded onto Metrichor EPI2ME analysis platform to perform real time identification of N. gonorrhoeae using WIMP workflow which aligns read sequence with the reference sequence in NCBI RefSeq database covering all the bacterial, viral and fungal genomes available in RefSeq (Juul et al., 2015).

### Example 6: PCR Amplicon Sequencing and Analysis

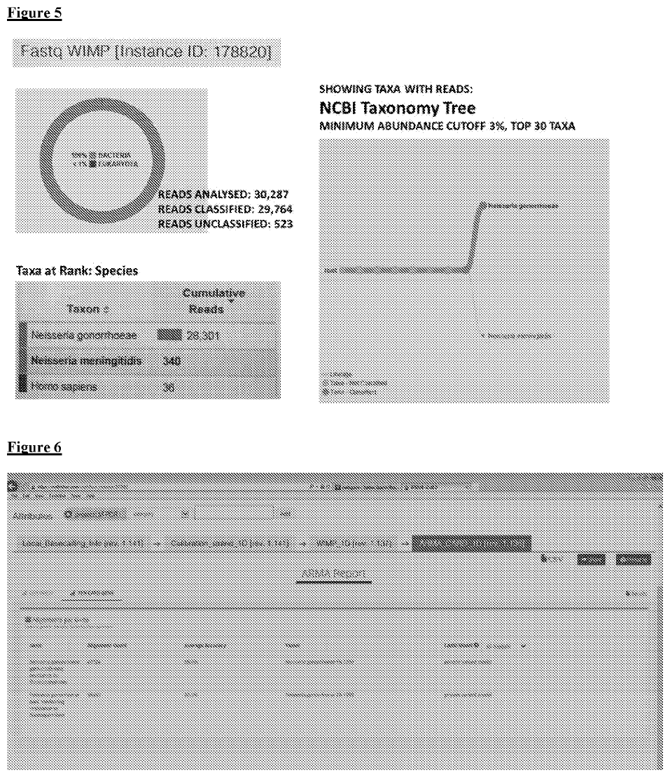

Nanopore amplicon sequencing was performed on MinION Mk I using SpotON Flow Cell Mk I 20 (R9.4) (Oxford Nanopore Technologies). A sequencing library was prepared from PCR amplicons with or without barcode using the Ligation Sequencing Kit 1D (SQK-LSK108) according to the manufacturer's instruction, except that 0.2 pmoles of DNA in the adapter ligation step was used. Nanopore sequencing was performed by local base-calling method using the script NC_48Hr_Sequencing_Run_FLO-MIN106 SQK-LSK108.py. To perform real time detection and antibiotic resistance profiling, locally base-called 1D reads were uploaded onto Metrichor EPI2ME analysis platform and analysed using AMRA workflow, of which the WIMP 1D Rev 1.137 component aligns each read sequence with the reference sequence in NCBI RefSeq database for pathogen identification and ARMA CARD 1D Rev 1.136 component scans the CARD database for antimicrobial resistance prediction. The analysed reads were automatically download back to the local computer. Examples of results are shown in FIGS. 5 and 6.

### Example 7: Comparing the Results Derived from the Sequencing with and without Targeted PCR Amplification

Following the sequencing of the DNA libraries, target read counts were compared between the sequencing library constructed with sample DNA without PCR amplification such as in Example 5 and the library constructed with the PCR amplicons derived from PCR amplification on the sample DNA such as in Example 6, as shown in Table 1. The library without PCR amplification produced 81 NG read counts out of a total 105,305 reads, while the library with PCR amplification produced 95,766 NG reads out of 118,563 reads. Furthermore, among the NG reads, the library with PCR amplification produced 40,324 gyrA and 36,340 parC reads, which were identified as fluoroquinolone resistance by protein variant model. However, the library without targeted PCR amplification produced none of gyrA and parC reads, which cannot tell if the identified microorganism is antibiotic resistance. This clearly demonstrated that using a targeted PCR on a single gene responsible for antibiotic resistance can allow for the pathogen to be identified and its antibiotic resistance profile determined simultaneously.

TABLE 1
Comparison of sequencing results with or without targeted PCR
amplification

| | | Run | | Base | | CARD resistance |
|---|---|---|---|---|---|---|
| Sample | Targeted | time | Reads | yields | N. gonorrhoeae | count:gene:antibiotic; model |
| ID | PCR | (Hour) | analysed | (Mb) | read counts | (Avg. accuracy) |
| 3020 | No | 6.5 | 105,305 | 240.3 | 81 | 0:gyrA:fluoroquinolones:protein |
| | | | | | | variant |
| | | | | | | 0:parC:fluoroquinolones:protein |
| | | | | | | variant |
| | Yes | 2.5 | 118,563 | 140.4 | 95,766 | 40,324:gyrA:fluoroquinolones: |
| | | | | | | protein variant (85%) |
| | | | | | | 36,340:parC:fluoroquinolones: |
| | | | | | | protein variant (86%) |

### Example 8: Additional Data Demonstrating Use of Single Gene Sequencing for Simultaneous Diagnosis and Antibiotic Resistance Detection for Sexually Transmitted Infections

Vulvo-vaginal swab samples were collected by clinicians using Xpert® CT/NG Patient-Collected Vaginal Swab Specimen Collection kit (Cepheid) and DNA from these samples prepared using PureLink genomic DNA Mini Kit (Thermo Fisher Scientific) according to manufacturer's instructions. An aliquot of DNA preparation was used for PCR amplification. Neisseria gonorrhoeae (NG), Trichomonas vaginalis (TV), Mycoplasma genitalium (MG) and Chlamydia trachomatis (CT) infections were PCR-screened using gene targets and primers listed in Table 2.

TABLE 2
Gene targets and primers used by real time PCR and long read sequencing
Pathogen, target, primer and probe
Trichomonas vaginalis
T. vaginalis-specific repeat DNA fragment
TV_F: AAAGATGGGTGTTTTAAGCTAGATAAGG (SEQ ID NO: 17)
TV_R: TCTGTGCCGTCTTCAAGTATGC (SEQ ID NO: 18)
TV_P: [6FAM]AGTTCATGTCCTCTCCAAGCGTAAGT[BHQ1]
Nitroreductase family protein (ntr6) (SEQ ID NO: 19)
TV_NTR6_BC_F:
5' TTTCTGTTGGTGCTGATATTGCCTTCATTGAATTTATTCGTTCAAAATT
(SEQ ID NO: 15)
TV_NTR6_BC_R:
5'ACTTGCCTGTCGCTCTATCTTCTTATTCAATGTATGTAACCTTTCTAA
(SEQ ID NO: 16)
Mycoplasma genitalium
MgPa adhesin gene
MG_F: GAGAAATACCTTGATGGTCAGCAA (SEQ ID NO: 20)
MG_R: GTTAATATCATATAAAGCTCTACCGTTGTTATC (SEQ ID NO: 21)
MG_P: [6FAM]ACTTTGCAATCAGAAGGT[MGBNFQ] (SEQ ID NO: 22)
23S ribosomal RNA
Mg 23S_BC_1992F:
5' TTTCTGTTGGTGCTGATATTGCCCATCTCTTGACTGTCTCGG (SEQ ID
NO: 11)
Mg 23S_BC_2679R:
5' ACTTGCCTGTCGCTCTATCTTCTCCTCTCGTACTAGAAGCAAAG (SEQ
ID NO: 12)
Chlamydia trachomatis
The cryptic plasmid
CT_F: CATGAAAACTCGTTCCGAAATAGAA (SEQ ID NO: 23)
CT_R: TCAGAGCTTTACCTAACAACGCATA (SEQ ID NO: 24)
CT_P: [6FAM]TCGCATGCAAGATATCGA[MGBNFQ] (SEQ ID NO: 25)
Major outer membrane protein 1 (omp1)
CT_OMP1_BC_F:
5' TTTCTGTTGGTGCTGATATTGCTTTGCCGCTTTGAGTTCTGCT (SEQ ID
NO: 7)
CT_OMP1_BC_R:
5' ACTTGCCTGTCGCTCTATCTTCCAATACCGCAAGATTTTCTAGATTTC
(SEQ ID NO: 8)
Neisseria gonorrhoeae
opa gene (opa)
NG_F: TTGAAACACCGCCCGGAA (SEQ ID NO: 26)
NG_R: TTTCGGCTCCTTATTCGGTTTGA (SEQ ID NO: 27)
NG_P: [6FAM]CCGATATAATCCGCCCTTCAACATCAG[BHQ1] (SEQ ID NO:
28)
gyrA gene
NG_gyrA_BC_F:
5' TTTCTGTTGGTGCTGATATTGCATCCGCCACGACCACAAATT (SEQ ID
NO: 3)
NG_gyrA_BC_R:
5' ACTTGCCTGTCGCTCTATCTTCATATTGGACAGTGCGACGGC (SEQ ID
NO: 4)

Real-time PCR was performed using Applied Biosystems 7500 Fast Real-Time PCR System in a volume of 10 μl containing 5 μl of TaqMan™ Fast Universal PCR Master Mix, 1 μl of 10× Exogenous Internal Positive Control (IPC) Mix, 0.2 μl of 50×IPC DNA, 250 nM each of primers, 100 nM each of probes and 50 ng of template DNA. Cycling parameters: 95° C., 10 min; 40 cycles of 95° C. for 15 s; 60° C. for 1 min. Of 200 PCR-screened samples, 11 had MG infection, 19 TV, 7 CT, 2 CT and TV dual infection, 1 NG and CT, 1 NG and TV, 1 NG, CT and TV triple infections and 1 CT, TV and MG.

A single gene for each STI pathogen was selected to use as a target in singlex or triplex PCR targeted long-read sequencing for simultaneous identification and antibiotic resistance detection: NG gyrA responsible for NG fluoroquinolone resistance, TV ntr6 responsible for TV metronidazole resistance, and MG 23 S rRNA responsible for MG macrolide resistance. In addition, CT omp1 gene was used for identifying CT infection. Primers for targeted PCR amplification and barcoding are also included in Table 2. PCR reaction was performed in a 50 μl volume consisting of 25 μl LongAmp PCR mix (NEB), 0.2 μM each of PCR primers and 100 ng DNA template for singlex PCR, while triplex PCR used various primer concentrations: 0.15 μM each of NG primers, 0.3 μM each of MG primers and 0.15 μM each of CT primers. PCR condition was as follows: 95° C., 3 min; 35 cycles of 95° C., 30 s; 58° C., 30 s; 65° C., lmin 30 s; and one cycle of 65° C., 5 min. Following amplification, PCR amplicons were purified and subjected to a second PCR for barcoding using the PCR Barcoding kit [Oxford Nanopore Technologies (ONT)] according to manufacturer's instructions.

For singlex PCR targeted long-read sequencing, 64 samples including 51 positives, 12 negatives (3 for each pathogen) and an NG positive control were amplified by singlex PCR, barcoded and pooled in one sequencing library, which was constructed using Ligation Sequencing Kit—SQK-LSK108 (ONT). Long-read sequencing was performed using ONT MinION sequencer and sequencing data analysed on ONT Metrichor Epi2ME using ‘What's In My Pot (WIMP) program for pathogen identification.

A sequencing run of 10 hours produced 1,499,872 reads with total yield of 1.2 Gbases, of which 76% was workflow successful with average quality score 8.34 and average sequence length 816 bases. To identify MG, NG and CT, a total of 1,127,701 reads were analyzed by WIMP, resulting in 810,683 (72%) classified reads of which 43,491 (5.4%) were non-barcoded. To identify TV infections, Fastq Custom Alignment was used because of no entries of TV reference in the database and a total of 1,483,872 reads analyzed, producing 148,458 alignments with TV ntr6 sequence.

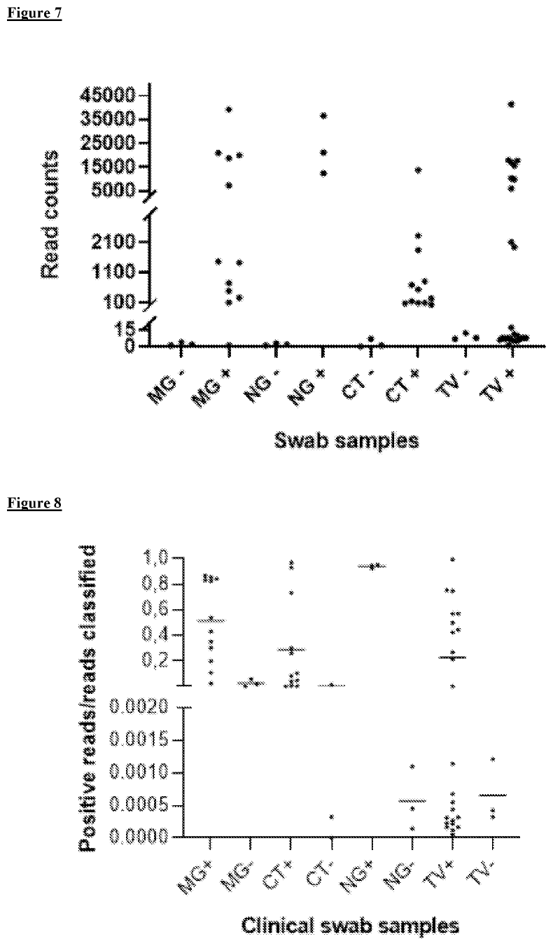

In singlex PCR-targeted long-read sequencing, a majority of PCR-positive samples yielded much higher pathogen specific read counts than PCR-negative samples, which had fewer than 20 reads (FIG. 7).

Ratios of pathogen-specific reads and read counts classified for each sample are shown in FIG. 8. By comparing these ratios, it was possible to identify through long-read sequencing that 3/3 NG samples, 11/12 of MG samples, 11/12 CT samples and 13/24 TV samples were appropriately called positive. In the 13/48 positive samples incorrectly called negative by long-read sequencing, pathogen loads were much lower, as evidenced by higher Ct values (FIG. 9).

In order to reduce both ‘sample-in to result time’ and cost, a triplex PCR was developed targeting NG, CT and MG in clinical samples and integrated it into long read sequencing, (integrating TV is scheduled for later). Gene targets, primers and samples for triplex PCR targeted long-read sequencing were the same as used in singlex PCR targeted long-read sequencing.

In triplex PCR-targeted long-read sequencing, 45 samples, including 3 with NG infection, 10 with MG and 11 with CT were amplified by triplex PCR, barcoded and pooled in one sequencing library, constructed using Ligation Sequencing Kit—SQK-LSK108 (ONT). Long-read sequencing was performed using ONT MinION sequencer and sequencing data analysed on ONT Metrichor Epi2ME using WIMP.

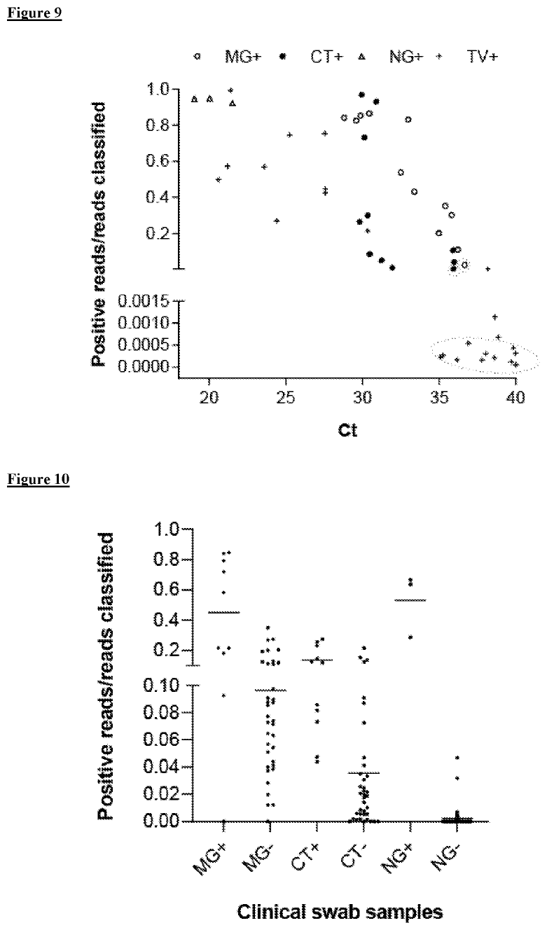

A 10 hours' run, with 56% workflow success, produced 811,677 reads with total yield of 268.9 Mbases, average quality score 6.74 and average sequence length 331 bases. In spite of this lower sequencing yield, 71,124 (15.6%) out of 454,863 reads were classified, of which 13,333 were non-barcoded. Ratios of pathogen specific reads to classified read counts (FIG. 10) enabled accurate identification of 3/3 NG, 8/10 MG and 11/11 CT infected samples. Furthermore, triplex PCR targeted long-read sequencing took less time to perform than a multiple singlex PCR approach with significant cost reduction.

To demonstrate use of a single gene for identifying and profiling antibiotic resistance of STIs simultaneously, long-read DNA sequences from each of three pathogens (NG, MG and TV) were extracted from read FASTQ files. Consensus sequences were constructed by aligning antimicrobial resistance associated sequence regions and accuracy of antimicrobial resistance prediction interrogated by manual BLAST comparing consensus sequence against reference sequences: Neisseria gonorrhoeae FA 1090 gyrA reference (NC_002946.2:c621189-618439), Mycoplasma genitalium strain G-37 23S ribosomal RNA gene (NR_077054.1) and Trichomonas vaginalis G3 nitroreductase family protein reference (TVAG_354010) respectively.

Manual BLAST search confirmed that all (3/3) of NG had fluoroquinolone-resistant mutations, 20% (2/10) of TV had mutations related to metronidazole resistance, and none of MG had macrolide resistance-associated mutations, as shown in Table 3.

TABLE 3
Antimicrobial resistance associated mutations* identified by singlex and
triplex PCR targeted long read sequencing.

| Genes and samples | Sequence of antimicrobial resistance region |
|---|---|
| NG gyrA | |
| Wild type | -TCCGCAGTTTACGAC- (SEQ ID NO: 29) |
| NG samples analysed | |
| NG_58, 59 and 60 samples | -TTCGCAGTTTACGCC- (SEQ ID NO: 30) |
| NG_64(positive control) | -TCCGCAGTTTACGAC- (SEQ ID NO: 29) |
| MG 23S rRNA | |
| Wild type | -CGGGACGGAAAGACC-(SEQ ID NO: 31) |
| MG samples analysed | |
| All MG samples | -CGGGACGGAAAGACC-(SEQ ID NO: 31) |
| TV ntr6 | |
| Wild type | -AATGCAAAAGCAGAC-(SEQ ID NO: 32) |
| TV samples analysed | |
| TV_17 and 34 samples | -AATGCAATAGCAGAC-(SEQ ID NO: 33) |
| Other 8 TV samples | -AATGCAAAAGCAGAC-(SEQ ID NO: 32) |

*One or more changes of bold bases in wild type cause antimicrobial resistance of strains

Using as examples, the gyrA gene responsible for NG fluoroquinolone resistance, ntr6 gene responsible for TV metronidazole resistance, and 23S rRNA gene responsible for MG macrolide resistance, a single DNA fragment (gene) responsible for pathogen antibiotic resistance can be used for simultaneous identification and antibiotic resistance detection of sexually transmitted infections through PCR targeted long-read sequencing

1. A method for simultaneously detecting a microorganism and for profiling antimicrobial resistance of the microorganism, comprising:
(a) preparing DNA from a sample suspected of containing the microorganism;
(b) conducting targeted amplification of a gene from the microorganism, wherein the gene is responsible for antimicrobial resistance and can identify the microorganism;
(c) sequencing the amplified products obtained in step (b) using long-read sequencing;
(d) comparing the sequence to reference sequences known to be susceptible or resistant to the antimicrobial; and
(e) identifying the microorganism and profiling the antimicrobial resistance of the microorganism.

2. The method of claim 1, wherein the sample is a blood, urine, saliva, faeces sample, or a swab, or a mixture thereof.

3. The method of claim 1 or 2, wherein the target gene is amplified using PCR, optionally performed in singlex and/or multiplex format.

4. The method of claim 3, wherein the PCR uses barcoded primers.

5. The method of claim 3 or 4, wherein the PCR amplicons are pooled and used to construct a sequencing library.

6. The method of any one of the preceding claims, wherein the amplified products of step (b) are purified prior to sequencing.

7. The method of any one of the preceding claims, where the long-read sequencing is nanopore sequencing.

8. The method of any one of the preceding claims, wherein the microorganism is a pathogenic bacteria, parasite or fungus.

9. The method of claim 8, wherein the microorganism is responsible for a sexually transmitted infection.

10. The method of claim 9, wherein the microorganism is Neisseria gonorrhoeae, Mycoplasma genitalium, Chlamydia trachomatis or Trichomonas vaginalis.

11. The method of claim 10, wherein:
(a) the microorganism is Neisseria gonorrhoeae and the target gene is gyrA;
(b) the microorganism is Mycoplasma genitalium and the target gene is 23S rRNA; or
(c) the microorganism is Trichomonas vaginalis and the target gene is Ntr6.

12. The method of any one of the preceding claims, wherein the sample is obtained from a subject suspected of having an infection with a microorganism.

13. A method for identifying a microorganism, comprising:
(a) preparing DNA from a sample suspected of containing the microorganism;
(b) conducting targeted amplification of a gene from the microorganism, wherein the gene can identify the microorganism;
(c) sequencing the amplified products obtained in step (b) using long-read sequencing;
(d) comparing the sequence to reference sequences; and
(e) identifying the microorganism.

14. The method of claim 13, wherein the microorganism is Chlamydia trachomatis and the target gene is omp1.

15. A method for profiling the antimicrobial resistance of a microorganism, comprising:
(a) preparing DNA from a sample containing the microorganism;
(b) conducting targeted amplification of a gene from the microorganism, wherein the gene is responsible for antimicrobial resistance;
(c) sequencing the amplified products obtained in step (b) using long-read sequencing;
(d) comparing the sequence to reference sequences; and
(e) profiling the antimicrobial resistance of the microorganism.

16. A kit for use in the method of any one of the preceding claims, comprising reagents for amplification of one or more target genes and instructions for use.

17. The kit of claim 16, comprising primers for amplification of the one or more target genes.


SEQUENCE LISTING (33 sequences)

SEQ ID NO: 1 - DNA, 20 bp, Artificial Sequence
  NG_gyrA_F primer
atccgccacgaccacaaatt

SEQ ID NO: 2 - DNA, 20 bp, Artificial Sequence
  NG_gyrA_R primer
atattggacagtgcgacggc

SEQ ID NO: 3 - DNA, 42 bp, Artificial Sequence
  NG_gyrA_BC_F primer
tttctgttggtgctgatattgcatccgccacgaccacaaatt

SEQ ID NO: 4 - DNA, 42 bp, Artificial Sequence
  NG_gyrA_BC_R primer
acttgcctgtcgctctatcttcatattggacagtgcgacggc

SEQ ID NO: 5 - DNA, 21 bp, Artificial Sequence
  CT_OMP1_F primer
tttgccgctttgagttctgct

SEQ ID NO: 6 - DNA, 26 bp, Artificial Sequence
  CT_OMP1_R primer
caataccgcaagattttctagatttc

SEQ ID NO: 7 - DNA, 43 bp, Artificial Sequence
  CT_OMP1_BC_F primer
tttctgttggtgctgatattgctttgccgctttgagttctgct

SEQ ID NO: 8 - DNA, 48 bp, Artificial Sequence
  CT_OMP1_BC_R primer
acttgcctgtcgctctatcttccaataccgcaagattttctagatttc

SEQ ID NO: 9 - DNA, 20 bp, Artificial Sequence
  Mg 23S_1992F primer
ccatctcttgactgtctcgg

SEQ ID NO: 10 - DNA, 19 bp, Artificial Sequence
  Mg 23S_2679R primer
tctcgtactagaagcaaag

SEQ ID NO: 11 - DNA, 42 bp, Artificial Sequence
  Mg 23S_BC_1992F primer
tttctgttggtgctgatattgcccatctcttgactgtctcgg

SEQ ID NO: 12 - DNA, 44 bp, Artificial Sequence
  Mg 23S_BC_2679R
acttgcctgtcgctctatcttctcctctcgtactagaagcaaag

SEQ ID NO: 13 - DNA, 27 bp, Artificial Sequence
  TV_NTR6_F primer
cttcattgaatttattcgttcaaaatt

SEQ ID NO: 14 - DNA, 26 bp, Artificial Sequence
  TV_NTR6_R
ttattcaatgtatgtaacctttctaa

SEQ ID NO: 15 - DNA, 49 bp, Artificial Sequence
  TV_NTR6_BC_F primer
tttctgttggtgctgatattgccttcattgaatttattcgttcaaaatt

SEQ ID NO: 16 - DNA, 48 bp, Artificial Sequence
  TV_NTR6_BC_R primer
acttgcctgtcgctctatcttcttattcaatgtatgtaacctttctaa

SEQ ID NO: 17 - DNA, 28 bp, Artificial Sequence
  T. vaginalis specific repeat DNA fragment forward primer
aaagatgggtgttttaagctagataagg

SEQ ID NO: 18 - DNA, 22 bp, Artificial Sequence
  T. vaginalis specific repeat DNA fragment reverse primer
tctgtgccgtcttcaagtatgc

SEQ ID NO: 19 - DNA, 26 bp, Artificial Sequence
  T. vaginalis specific repeat DNA fragment probe
  misc_feature (1)..(1): 6FAM
  misc_feature (26)..(26): BHQ1
agttcatgtcctctccaagcgtaagt

SEQ ID NO: 20 - DNA, 24 bp, Artificial Sequence
  MgPa adhesin gene MG_F primer
gagaaataccttgatggtcagcaa

SEQ ID NO: 21 - DNA, 33 bp, Artificial Sequence
  MgPa adhesin gene MG_R primer
gttaatatcatataaagctctaccgttgttatc

SEQ ID NO: 22 - DNA, 18 bp, Artificial Sequence
  MgPa adhesin gene MG probe
  misc_feature (1)..(1): 6FAM
  misc_feature (18)..(18): MGBNFQ
actttgcaatcagaaggt

SEQ ID NO: 23 - DNA, 25 bp, Artificial Sequence
  cryptic plasmid CT_F primer
catgaaaactcgttccgaaatagaa

SEQ ID NO: 24 - DNA, 25 bp, Artificial Sequence
  cryptic plasmid CT_R primer
tcagagctttacctaacaacgcata

SEQ ID NO: 25 - DNA, 18 bp, Artificial Sequence
  cryptic plasmid probe
  misc_feature (1)..(1): 6FAM
  misc_feature (18)..(18): MGBNFQ
tcgcatgcaagatatcga

SEQ ID NO: 26 - DNA, 18 bp, Artificial Sequence
  opa gene NG_F primer
ttgaaacaccgcccggaa

SEQ ID NO: 27 - DNA, 23 bp, Artificial Sequence
  opa gene NG_R primer
tttcggctccttattcggtttga

SEQ ID NO: 28 - DNA, 27 bp, Artificial Sequence
  opa gene NG probe
  misc_feature (1)..(1): 6FAM
  misc_feature (27)..(27): BHQ1
ccgatataatccgcccttcaacatcag

SEQ ID NO: 29 - DNA, 15 bp, Artificial Sequence
  NG gyrA antimicrobial reistance region WT
tccgcagtttacgac

SEQ ID NO: 30 - DNA, 15 bp, Artificial Sequence
  NG gyrA antimicrobial resistance region in samples 58, 59 and 60
ttcgcagtttacgcc

SEQ ID NO: 31 - DNA, 15 bp, Artificial Sequence
  MG 23S rRNA antimicrobial resistance region
cgggacggaaagacc

SEQ ID NO: 32 - DNA, 15 bp, Artificial Sequence
  TV ntr6 antimicrobial resistance region wild type
aatgcaaaagcagac

SEQ ID NO: 33 - DNA, 15 bp, Artificial Sequence
  TV ntr6 antimicrobial resistance region samples 17 and 34
aatgcaatagcagac